(19) United States
(12) Patent Application Publication
(10) Pub. No.: US 2022/0273352 A1
(43) Pub. Date: Sep. 1, 2022

(54) DEVICE AND METHOD FOR THERMAL MODULATION OF TISSUE

(71) Applicant: BlueXthermal, Inc., Cambridge, MA (US)

(72) Inventor: Reza Monazami, Cambridge, MA (US)

(21) Appl. No.: 17/653,122

(22) Filed: Mar. 1, 2022

Related U.S. Application Data
(60) Provisional application No. 63/155,236, filed on Mar. 1, 2021.

(51) Int. Cl. A61B 18/06; A61B 18/02

(52) U.S. Cl. CPC A61B 18/06; A61B 18/02; A61B 2018/0293; A61B 2018/00744

(57) ABSTRACT

Devices and methods for thermally modulating tissue are disclosed herein. Exemplary embodiments of the present technology can comprise a device including a probe and a chemical reactant supply fluidically coupled to the probe. The probe includes an elongated shaft member, a reaction chamber, a plurality of lumens each extending from a proximal end portion of the probe to the reaction chamber, and a tip at a distal terminus of the probe. The chemical reactant supply includes a first chemical reactant and a second chemical reactant. The tip is configured to penetrate tissue of a patient such that the reaction chamber is positioned adjacent a target region of the patient. The reaction chamber is configured to receive the first and second chemical reactants to cause an exothermic or endothermic reaction that in operation cools or heats, respectively, the target region.

### CROSS-REFERENCE TO RELATED APPLICATION(S)

This application claims priority to and the benefit of U.S. Provisional Patent Application No. 63/155,236, filed Mar. 1, 2021, the disclosure of which is incorporated herein by reference in its entirety.

### TECHNICAL FIELD

The present invention relates generally to a device and method for thermal modulation of tissue. More specifically, the present invention relates to a device and method for chemical reaction-induced heating and cooling of tissue using a probe inserted into the tissue.

### BACKGROUND

Methods for heating and cooling tissue are known and include, for example, hyperthermia methods such as thermal ablation. Thermal ablation uses heat to cause damage to cancer cells of the tissue, and uses radio waves, microwaves, ultrasound waves, and other forms of energy to heat a target region. See for example U.S. Pat. No. 9,802,063. Likewise, methods for cooling tissue are known, and include, for example, cryogenic tissue remodeling methods which deliver cooling liquid to the tissue. See for example U.S. Pat. No. 10,363,080. The efficiency of these heating and cooling methods is compromised due to delivery and transfer of the heating and cooling to the actual target region. In addition, these tissue heating and cooling methods cause unwanted heating and cooling of the surrounding healthy tissues. A pressing need therefore exists to develop an improved device and method that allows efficient, regulated and precise thermal modulation of a target tissue within a patient in need of treatment using either a heating or cooling effect of the target tissue.

### SUMMARY

The present disclosure provides a device as described herein for cooling and heating a target tissue and a method of using that device. The device includes a probe having a chamber therein to receive chemical reactants that when reacted produce either an exothermic or endothermic chemical reaction. The reactants may mix within the chamber by introduction into the chamber and/or confinement within the chamber. The chamber may have a static mixer within the chamber. The chamber may have an active mixer within the chamber. According to one aspect, the probe may have an open tip. According to one aspect, the probe may have a closed tip. The chamber can be located at a discrete position within the interior of the probe, such as proximal to the probe tip, or it can span the length of the probe, as desired to provide a heating or cooling effect at one or more or various or continuous tissue contact locations. The probe may have one or more mixing chambers located within the probe, each with separate input conduits or lumens for providing reactants within the chamber. The probe is placed in contact with target tissue. The chemical reactants are introduced into the chamber. A chemical reaction takes place to cause either a heating or cooling effect depending upon the selected reactants. The probe itself can be used to treat tissue by subjecting the tissue to either a heating or cooling effect, or it can be used in conjunction with other treatment devices where heating or cooling of target tissue is desired, such as electronic devices, optical devices, lasers, biomedical devices and other devices where cooling and heating of a target tissue is desirable.

Aspects of the present disclosure are directed to a device and method for modulating the temperature of a target tissue, such as heating the target tissue to a treatment temperature such as to destroy the target tissue, or cooling the target tissue to a treatment temperature such as to destroy the target tissue. The device is configured to provide heating or cooling of a target tissue, such as a tissue surface area or a region or volume within a tissue. According to one aspect, the device for modulating the temperature of a target tissue includes a probe component including a chamber to receive a chemical reactant supply and a chemical reactant supply component. According to one aspect, the probe may include a pointed tip so as to facilitate entry of the probe through and into tissue. A probe with a pointed tip may be referred to herein as a needle. In an exemplary embodiment, the tip of the needle is closed. The chemical reactant supply component includes two or more containers where chemical reactants are stored. The chemical reactant supply component further includes a flow control system having conduits to control the flow and supply of the chemical reactants from the containers via the conduits into the probe component. When the chemical reactants meet within the chamber of the probe, chemical reaction occurs within the chamber and accordingly within the probe and heat is released to or absorbed from the tissue resulting in heating or cooling of the tissue. According to one aspect, chemical reactants are subsequently added to the reaction chamber for a second reaction within the probe and where heat is released to or absorbed from the tissue resulting in heating or cooling of the tissue. According to one aspect, reaction products are removed from the chamber before reactants are added for a subsequent reaction. Chemical reactants may then be introduced into the chamber again so as to facilitate heating or cooling of tissue. In this manner, a first target tissue location may be treated with either heating or cooling, the probe is then located at the same or a second target tissue location and reactants are again added to the chamber where they react and facilitate heating or cooling of the same or second target tissue location. According to one aspect, the first reaction product or products may be removed or extracted or flushed from the chamber and the probe via an inflow conduit and an outflow conduit connecting the chamber, prior to subsequent introduction of reactants for heating or cooling.

These and other embodiments will become apparent from the following Detailed Description.

### BRIEF DESCRIPTION OF THE DRAWINGS

FIG. 1 illustrates in schematic a device for thermal modulation according to an exemplary embodiment of the present disclosure.

FIGS. 2A and 2B illustrate in schematic an exemplary needle probe component of the device for thermal modulation. FIG. 2A depicts the needle probe shaft member showing a single inner lumen and a sharpened distal closed end extending into target tissue. FIG. 2B is a cross-sectional view of the needle probe shaft member showing a single channel or lumen.

FIGS. 3A and 3B illustrate in schematic an exemplary needle probe component of the device for thermal modulation. FIG. 3A depicts is a partial transparent view of the needle probe shaft member showing multiple inner lumens with opening ports and a sharpened distal closed end extending into target tissue. FIG. 3B is a cross-sectional view of the needle probe shaft member showing multiple lumens.

FIG. 4 illustrates in schematic a perspective view of a device for thermal modulation according to another exemplary embodiment of the present disclosure.

FIG. 5 is a flowchart schematically illustrating a method for treating a target tissue according to an exemplary embodiment of the present disclosure.

FIG. 6 illustrates in schematic an embodiment of a probe or needle with two reaction chambers and conduits connecting the reaction chambers.

FIG. 7 depicts a partial view of the probe or needle similar to that of FIG. 6 and with a common first conduit and a common second conduit connecting the first and second reaction chambers.

The figures should be understood to present illustrations of embodiments of the invention and/or principles involved. As would be apparent to one of skill in the art having knowledge of the present disclosure, other devices, methods, and particularly equipment used in heating or cooling devices, temperature sensors, microchannels, and/or thermoelectric elements, will have configurations and components determined, in part, by their specific use. Like reference numerals refer to corresponding parts throughout the several views of the drawings.

### DETAILED DESCRIPTION

Aspects of the present disclosure are directed to a device for modulating the temperature of a target tissue. According to one aspect of the present disclosure, a temperature modulating device is disclosed that is configured to provide heating/cooling to a target tissue or region. According to one aspect, the heating or cooling is accomplished by chemical reactants reacting within a reaction chamber within the device that contacts tissue to produce either an exothermic reaction or a endothermic reaction. According to one aspect, the device for modulating the temperature of a target tissue includes a probe component and a chemical reactant supply component. In one embodiment, chemical reactants are stored in one or more containers of the chemical reactant supply component. In one embodiment, the chemical reactant supply component includes a flow control system and conduits to control the flow and supply of the chemical reactant from the containers via the conduits into the probe component where the chemical reactants react in the probe component resulting in heating or cooling of a target tissue.

According to one aspect, the probe has an elongated shaft member, a distal end, a proximal end and a reaction chamber within the interior of the probe. The chamber can be located at a discrete position within the interior of the probe, such as proximal to the probe tip, or it can span the length of the probe, as desired to provide a heating or cooling effect at one or more or various or continuous tissue contact locations. In one embodiment, the elongated shaft member has one or more inner lumens that are open at the distal end and that terminate within the probe at a mixing portion of the probe. The mixing portion of the probe can be the terminal portion of the one or more lumens or can be a chamber in fluid communication with the one or more lumens. According to one aspect, a single lumen is oriented along the longitudinal axis of the elongated shaft member. In an exemplary embodiment, the probe is a needle having a sharpened tip that can extend into the interior of a tissue. In alternative embodiments, the present disclosure contemplates that the elongated shaft member can be catheter, cannulas and other needle-like members having an equivalent elongated shaft member with one or more, two or more, three or more or a plurality of inner lumens for passage of chemical reactants. In one embodiment, the elongated shaft member has multiple coaxial inner lumens and each lumen is open at both the distal end and the proximal end. The multiple coaxial lumens are oriented along the longitudinal axis of the elongated shaft member. In one embodiment, the probe is a needle having multi-inner lumens. In one embodiment, the multi-inner lumens have open ports at both distal and proximal ends such that chemical reactants can travel and reach a chamber in which the reactants are mixed. One skilled in the art can choose an elongated shaft member having different configurations of single or multiple lumens, or an elongated shaft members having different shaped distal end such as tapered, sharpened, round or blunt as desired.

According to one aspect, the chemical reactant supply component of the device includes containers and conduits. In one embodiment, the containers store the chemical reactants. In one embodiment, the conduits operatively connect the containers to the probe. Additional elements can be included in the chemical reactant supply component of the device as described herein for flow control and data manipulation. In some embodiments, the conduits are single, multiple, segmented, branched, or are otherwise configured to allow desired passage or flow control of chemical reactants or reaction wastes. In one embodiment, one or more conduits are connected to the proximal end of the probe where the chemical reactants travel to and pass through the one or more inner lumens of the shaft of the probe and reach into the interior of the reaction chamber within the distal end of the shaft. In an alternative embodiment, the one or more conduits extend all the way from the proximal end of the shaft via one or more corresponding inner lumens of the shaft to the distal end of the shaft where the chemical reactants travel to and directly reach into the interior of a reaction chamber. In one embodiment, a single inner lumen and a single connected conduit is used to flow in or flow out one or more chemical reactants or reaction waste. In certain embodiments, one or more inner lumens and one or more connected conduits in any desired combination are used to flow in or flow out one or more chemical reactants or reaction waste. Reaction waste includes reaction products such as vapor.

According to one aspect, the device and method according to the present disclosure rely on positioning the reaction chamber at the distal end of the probe at, near or within a target tissue. In certain embodiments, the target tissue is a tissue of a patient, whether human or animal, in need of treatment. The target tissue may be located anywhere in the body where needle probe treatment may be beneficial. For example, the target tissue can be a solid tumor within an organ of the body, including but not limited to the liver, kidney, lung, bowel, stomach, pancreas, breast, prostate, uterus, muscle, or brain. The target tissue may be identified using conventional imaging techniques capable of elucidating a target tissue, e.g. a solid tumor, such as ultrasonic scanning, magnetic resonance imaging (MRI), computer-assisted tomography (CAT), fluoroscopy, nuclear scanning, or the like. High-resolution ultrasound or CT can be employed to monitor the size and location of the tumor or other lesion being treated, intraoperatively or externally.

According to one aspect, sensors are used to assist with the identification of the target tissue. For example, sensors are used to sense tissue parameters such as electrical impedance, temperature, pressure, and optical characteristics. In one embodiment, a sensor is disposed separately from the probe device. In another embodiment, a sensor is disposed together with the probe device. For example, a sensor can be disposed anywhere with the probe device. In one embodiment, a sensor is disposed in the interior of the reaction chamber of the probe. One or more sensors may be used in any desirable combination and disposition with the probe.

In one embodiment, the chemical reactant supply component of the device has a flow control system operatively connected to the conduits. In one embodiment, the flow control system includes an electric power source, along with a circuit having a processor for controlling the rate, duration and intensity of the flow applied by the device in response to actuation of an input. In one embodiment, the chemical reactants are fluidly and operatively coupled to the probe where the reaction chamber at the distal end of the probe is positioned at or near a target tissue of the patient so that an outer surface of the reaction chamber is at, within or near the target tissue. In such an embodiment, when the chemical reactants meet in the reaction chamber, either exothermal or endothermal reactions are generated in the reaction chamber and heat is released to or absorbed from the target tissue, resulting in heating or cooling of the target tissue. In certain embodiments, the flow control system includes one or more valves. In some embodiments, the valves may be disposed along the conduits so as to limit the rate and flow, temperature, time, rate of temperature change, or other heating or cooling characteristics. The valves can be powered electrically via a power source, per the direction of the processor. An exemplary power source comprises a rechargeable or single-use battery.

Processors suitable to be included in the flow control system of the device are known to one skilled in the art. An exemplary processor comprises a programmable electronic microprocessor embodying machine readable computer code or programming instructions for implementing one or more of the mode of action of the methods described herein. In one embodiment, the microprocessor is coupled to a memory (such as a non-volatile memory, a flash memory, a read-only memory (“ROM”), a random access memory (“RAM”), or the like) storing the computer code and data to be used thereby, and/or a recording media (including a magnetic recording media such as a hard disk, a floppy disk, or the like; or an optical recording media such as a CD or DVD) may be provided. Suitable interface devices (such as digital-to-analog or analog-to-digital converters, or the like) and input/output devices (such as USB or serial I/O ports, wireless communication cards, graphical display cards, and the like) may also be used. A wide variety of commercially available or specialized processor structures may be used in different embodiments, and suitable processors may make use of a wide variety of combinations of hardware and/or hardware/software combinations. For example, processor may be integrated on a single processor board and may run a single program or may make use of a plurality of boards running a number of different program modules in a wide variety of alternative distributed data processing or code architectures.

Without being bound by scientific theory, when the device for modulating the temperature of a target tissue is activated when the chemical reactants meet in the reaction chamber, in some embodiments, a first chemical reactant flows into the reaction chamber, followed by a second chemical reactant. In other embodiments, two chemical reactants flow into the reaction chamber simultaneously. The first and second chemical reactants are flowed in at ambient or starting temperature. When two reactants meet in the reaction chamber, exothermal or endothermal reactions happen depending on the type of reactants. In exothermal reaction, heat is released from the reaction chamber into the surrounding target tissue, resulting in heating the target tissue to a temperature that is higher than its starting temperature. In endothermal reaction, heat is absorbed from the surrounding target tissue into the reaction chamber, resulting in cooling of the target tissue to a temperature that is lower than its starting temperature.

According to one aspect, the probe is operatively connected to, with, or by, and/or operatively contacted to, with, or by the conduits of the flow control system. The flow control system may be fixed or removably connected to the probe as desired. According to one aspect, the probe and the flow control system may be fashioned within a unitary housing or may be fashioned to be a unitary structure. According to one aspect, the probe and the flow control system may be separate units or modules that are operatively connected to each other or are integral with each other and may be within a unitary housing.

In one embodiment, the proximal end of the needle shaft can have flow control connectors for inflow (injection) or outflow (aspiration) secured or removably attached through conduits such as tubing, and the flow can be controlled by a controller.

The general utility of the probe is to heat or cool the target tissue with the reaction chamber which may be located at the tip of the probe and/or at two or more or a plurality of reaction chambers located along the length of the probe with each reaction probe having input and output conduits or channels for input and removal of reactants and reaction products. In one embodiment, the shaft can be made of or coated with a material that prevents heat, e.g., from the reaction waste, to dissipate above or below the reaction chamber. In this manner, the heating or cooling effect can be localized to the reaction chamber and the tissue contacting the portion of the probe where the chamber is located. Exemplary material having sufficient heat conductivity for making the reaction chamber includes high or relatively high thermal conductivity materials such copper, silver, stainless steel, titanium, aluminum or other materials, alloys, or composite material. Gold, silver, graphene and other materials or alloys can be used as coating material for the reaction chamber or the shaft. The material used for the shaft can be the same as the material used for the reaction chamber or different depending on the application and the requirements of the thermal environment. Exemplary material having low thermal conductivity so as to concentrate or retain heat at or within the reaction chamber includes low thermal-conductivity materials such as plastics, nylons, Teflon, and specific metal alloys or composite materials can be used as insulating materials for the section above and below the reaction chamber. If the temperature drop caused by the reaction is very large then the reaction chamber and/or the needle wall can be both made of a low thermal-conductivity material or materials. The small thickness of the reaction chamber and needle wall makes it possible for the heat to flow in a radial direction while imposing significant thermal resistance in the longitudinal direction even for the case where the reaction chamber and/or the needle walls are made from a low thermal-conductivity material. In this manner, the probe can heat or cool a precisely targeted region of the tissue, i.e., only the intended region will be heated or cooled, reducing injury of unwanted region. The material prevents the shaft from overheating or overcooling during normal use. The shaft material is selected such that it has the ability to prevent unwanted loss or over heating of the tissue, as desired. It is to be understood that the device may be utilized to maintain the target tissue at a constant temperature or may lower and/or raise the temperature of the target tissue according to a desired temperature profile.

According to one aspect, the device of the present disclosure may be used to heat or otherwise increase the temperature of a target tissue. According to this objective, when the device is activated and chemical reactants flow in a direction from the proximal end of the shaft to the distal end of the shaft and reaches the reaction chamber, the reaction chamber heats or generates heat relative to its ambient or starting temperature, i.e., the temperature of the reaction chamber increases. In this manner, the temperature of the target tissue may be increased relative to its ambient temperature or starting temperature. When used in this operating mode to heat a target tissue, the needle probe device may not be subjected to heat removal from the target. However, it is to be understood that the needle may be removed or the chemical reactants be regulated to be operational in order to achieve a desired temperature profile of the target tissue, including lowering the temperature from an ambient or starting temperature and then raising the temperature.

According to one aspect, the device of the present disclosure may be used to cool or otherwise decrease the temperature of a target tissue. According to this objective, when the device is activated and chemical reactants flow in a direction from the proximal end of the shaft to the distal end of the shaft and reach the reaction chamber, the reaction chamber cools or absorbs heat relative to its ambient or starting temperature, i.e., the temperature of the reaction chamber decreases. In this manner, the temperature of the target tissue may be decreased relative to its ambient temperature or starting temperature. When used in this operating mode to cool a target tissue, the needle probe device may not be subjected to cold removal from the target. However, it is to be understood that the needle may be removed or the chemical reactants be regulated to be operational in order to achieve a desired temperature profile of the target tissue, including raising the temperature from an ambient or starting temperature and then lowering the temperature.

According to one aspect of the present disclosure, the device may be used to decrease and/or increase the temperature of the reaction chamber and therefore the target tissue in contact therewith according to a desired temperature profile using alternative flow of chemical reactants when desired. By changing the chemical reactants, the reaction chamber portion of the device may precisely cool or heat the target tissue in contact with the chemical reaction chamber according to a desired temperature profile over a desired period of time.

In general, the chemical reactants may be altered from one type to the other to repeatedly heat or cool the target tissue, or vice versa. The reactants may be removed from the reaction chamber in between separate heating and/or cooling steps.

An advantage of the device is the precision with regard to the desired duration and degree of temperature modulation of the target tissue. Another advantage of the device is the ability to modulate precisely a small sized target tissue, e.g., having a dimension of under 1 cubic millimeter, which is about 1000 fold less as compared to other conventional tissue heating or cooling devices or methods. An exemplary volume of the target tissue ranges from 1 cubic micron to 5 cubic centimeters, in particular 2 cubic microns to 1 cubic centimeter.

According to one aspect, a plurality of the probe device may be utilized together to achieve heating or cooling as described herein of a target tissue with a given region or area. In the heating mode, with each reaction chamber of the device generating heat within a given region or area, the device is capable of heating a desirable sized target tissue. In the cooling mode, with each reaction chamber of the device absorbing heat from a given region or area, the device is capable of cooling a desirable sized target tissue. According to one aspect, each probe in the plurality may either heat or cool tissue providing heating and/or cooling within a desired tissue surface area. In certain embodiments, each needle probe may have its own flow control system or a single unitary flow control system may control flow of reactants into one or more probes within the plurality of probes.

The probes or needles may be ordered or placed or positioned in an array format in rows and columns or in any desired pattern to achieve a desired objective which is to heat or cool a tissue in contact with the reaction chambers of the probe. For example, a device as described herein may include from 2 to 200 probes or needles arranged within the surface area of a thermally conductive plate or film or support as desired. Depending upon the number of probes or needles, the probes or needles may be arranged in a square pattern or a rectangular pattern or circular pattern or other pattern upon the surface area of or within the tissue to be heated or cooled. It is to be understood the term “probe” as used herein may refer to a needle.

Each probe may be operated independently to achieve different heating or cooling of different locations or positions of a target tissue to create thermal injury or all probes may be operated simultaneously to achieve heating or cooling of a target tissue within a given volume or surface area of tissue to create thermal injury. According to one aspect, point-wise thermal injury localized to the needle or probe is induced or created to a single region or multiple regions inside the target tissue. If a plurality of probes or needles are used, continued heating or cooling of each needle or probe may cause thermally affected regions may merge or collide with each other. According to one aspect, a short pulse is used to confine heating or cooling to the vicinity of the source of the heating or cooling pulse, and so only a region adjacent to or localized to the reaction chamber is affected.

Subsets of probes may be operated independently to achieve different heating or cooling of different locations on a target tissue.

According to one aspect, the device has applications in tumor ablation, pain management, cosmetic applications. It should be appreciated that various embodiments of the present invention device may be applied to and/or be utilized with a wide range of applications as desired, needed or required.

### I. Device for Thermal Modulation of Tissue

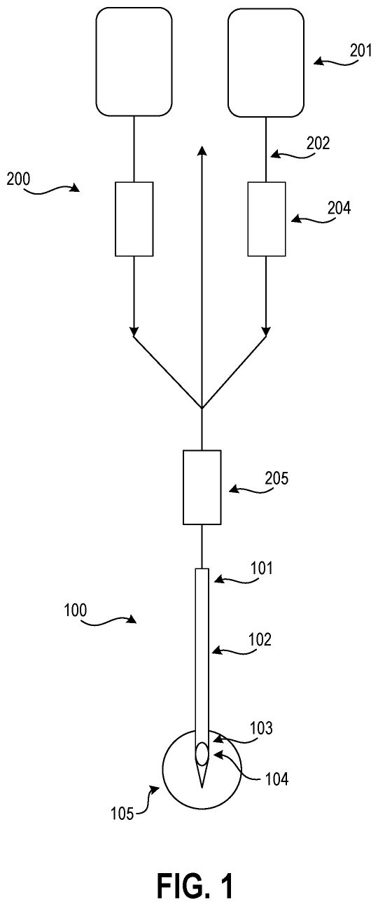

As illustrated in FIG. 1, the device according to the present disclosure includes a needle probe component (100) and a chemical reactant supply component (200). In one embodiment, the chemical reactant supply component includes containers (201) for storing the chemical reactants. In one embodiment, the chemical reactant supply component includes a flow control system (204) and conduits (202, 203) to control the flow and supply of the chemical reactant from the containers via the conduits into the needle probe component where the chemical reactants react in the needle probe component resulting in heating or cooling of a target tissue.

As further illustrated in FIG. 1, a needle probe component (100) of the device according to the present disclosure includes an elongated shaft member (102), a distal end (103), a proximal end (101) and a reaction chamber (104) positioned in the interior of the needle probe near the distal end. The needle probe is inserted into the surrounding tissue (105) which may be a tumor or tumor mass. In certain embodiments, the elongated shaft member has single or multiple inner lumens. In some embodiments, multiple inner lumens are configured coaxially. In some embodiments, multiple lumens are configured in parallel within the interior of the elongated shaft member. The inner lumen opens at both the distal and proximal end of the elongated shaft member. The present disclosure contemplates alternative needle shaft members including catheter, cannulas and other needle-like devices having similar rigid or flexible property as a needle shaft member or as desired. In one embodiment, the reaction chamber and the elongated shaft member are fashioned as a unitary integral member with the reaction chamber within the interior of the elongated shaft member. According to some embodiments, the probe (or needle) may have one or more, two or more, three or more or a plurality of reaction chambers position along the length of the probe (or needle) so as to create a pattern of heating or cooling (i.e., thermal injury) along the length of the probe (or needle), such that there may be two or more, three or more or a plurality of target tissue sites being treated. Description of a single reaction chamber herein may apply to one or more of additional reaction chambers if present in a particular embodiment.

The containers (201) of the chemical reactant supply component according to the present disclosure are configured to store the chemical reactants. Typically, each chemical reactant is stored in an individual container. The containers are made of materials that will not react with the chemical reactants. In certain embodiments, the containers are made with plastic, and/or composite materials that are compatible with critical parameters such as reactivity, stability, ease of transport and/or storage with the chemical reactants, and sterilization and the like. The materials for the containers include, but not limited to, polyethylene (PET), nylon, PE (cross linked and other polyolefin), polyurethane, polyvinyl chloride (PVC), and composite-like materials (polyamide/polyurethane composite), polyurethane TECO polymers, poly(tetrafluoroethane) or poly(tetrafluoroethylene) (PTFE), PEBAX, HYTREL, polyimide, and braided polyimide or composites thereof. The container material can also be stainless steel or other metals or alloys or composite materials with or without specific coating protecting the container wall from being affected by or contaminating the chemical reactants. In some embodiments, the containers are connected directly or indirectly to the needle probe via conduits, such as tubes. The conduits may be connected to the proximal end or the distal end of the needle probe as desired. Chemical reactants travel inside the conduit until they reach the interior of the reaction chamber. In an exemplary embodiment, one tube is used to deliver one type of chemical reactant to avoid contamination or reaction between chemical reactants prior to reaching the reaction chamber. In another exemplary embodiment, different tubes are used for inflow and outflow of chemical reactants or reaction waste.

In some embodiments, conduits run along the inner lumen along the shaft longitudinal axis. In a single lumen shaft embodiment, multiple conduits can run along the inner lumen along the shaft longitudinal axis. In some embodiments, conduits may couple sensors (e.g., thermal, pressure, and/or impedance sensors) with signal data loggers through electrical wires, optical fibers, or any coupling cable for signal transmission known to the field.

In some embodiments, the flow control system may include extension members that connect, e.g., a quick pneumatic/electric connector to a controller and to the containers storing the chemical reactants.

The lumen of conduits, tubes, cannula, catheter or needle can be made of metal, plastic, and or composite materials that are compatible with critical parameters such as pressure, profile, flexibility, and compatibility with the chemical reactants, and sterilization and the like. In some embodiments, they may be able to incorporate wires and microcomponents such as sensors or optical systems. The materials for conduits, tubes, cannula, catheter or needle include, but not limited to, stainless steel, polyethylene (PET), nylon, rubber, PE (cross linked and other polyolefin), polyurethane, polyvinyl chloride (PVC), and composite-like materials (polyamide/polyurethane composite), polyurethane TECO polymers, poly(tetrafluoroethane) or poly(tetrafluoroethylene) (PTFE), PEBAX, HYTREL, polyimide, and braided polyimide.

### II. Needle Probe Component

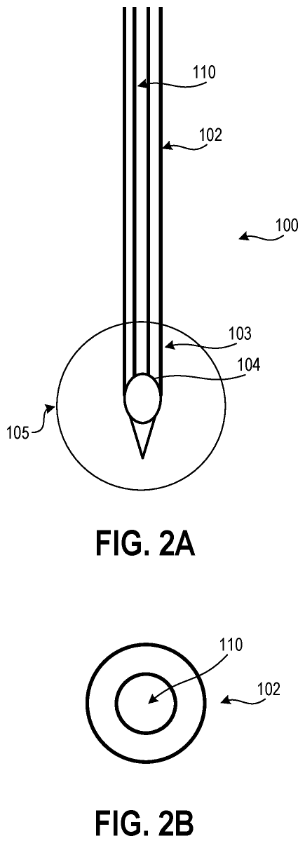

The needle probe according to the present disclosure as described herein has an elongated shaft member having one or more inner lumens, a proximal end, a distal end and a reaction chamber positioned within the interior of the probe near the distal end. FIG. 2A illustrates an embodiment of a partial view of the needle probe where the elongated shaft member (102) has a single lumen (110) along the longitudinal axis of the elongated shaft member. The reaction chamber (104) illustrated in schematic for ease of understanding as an oval is in the interior of the needle probe near the distal end (103) of the needle probe (100). The needle probe can be blunt, tapered or sharpened as desired to allow access into a target tissue. FIG. 2A further illustrates an embodiment where the tip of the distal end of the single lumen needle probe is a sharpened tip extending inwardly a distance into the surrounding tissue (105). FIG. 2B illustrates a cross-sectional view of the shaft member (102) having a single lumen (110). The single lumen has an opening port (not shown) that opens at the distal end to allow for desirable inflow and outflow of chemical reactants and reaction products.

The proximal end of the needle probe is operatively secured or attached to the conduit of the chemical supply component to allow passage of the chemical reactants into the inner lumen of the elongated shaft member of the needle probe.

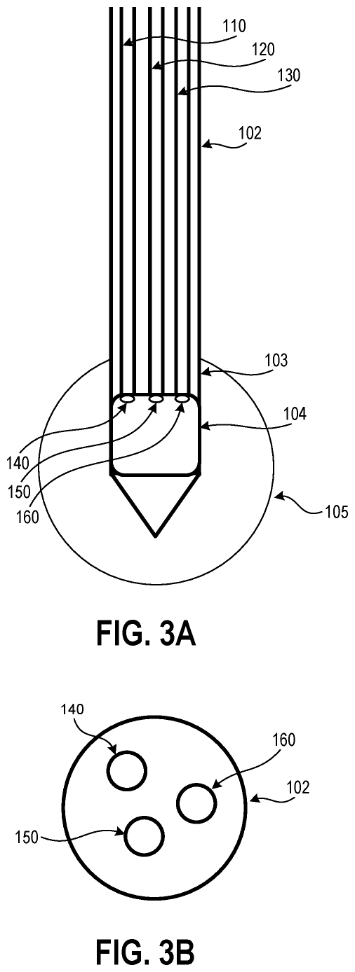

In some embodiments, multiple coaxial lumens can be configured to allow inflow of different chemical reactants into the reaction chamber with each lumen corresponds to a specific chemical reactant. FIG. 3A illustrate an embodiment of a partial view of a needle probe where the shaft (102) has multiple coaxial inner lumens (110, 120 and 130). In this configuration, multiple lumens can allow different inflow of chemical reactants. One or more multiple lumens can also allow outflow of reaction products from the reaction chamber. FIG. 3B illustrates a cross-sectional view of the shaft (102) having multiple inner lumens (110, 120, and 130). The outflow reaction products are collected and disposed of as desired. The multiple lumens each has an opening port (140, 150, 160) that opens at the distal end to allow for desirable inflow and outflow of chemical reactants and reaction products. Opening ports are also on the corresponding proximal end (not shown). Each opening port corresponds to a lumen.

The needle probe according to the present disclosure as described herein has an elongated shaft member. In alternative embodiments, needle, catheter, cannula or the like can be configured to have an elongated shaft member and used interchangeably to direct the passage of chemical reactants from the chemical reactant supply to the desired tissue. The wall of the shaft lumen can be made of metal, plastic, and/or composite materials that are compatible with critical parameters such as pressure, profile, flexibility, and compatibility with the chemical reactants, and sterilization and the like.

In some embodiments, the needle probe is configured to be able to penetrate tissue. In some embodiments, tissue penetration is achieved by the shape of the tip of the probe. In such embodiments, the reaction chamber can have a shape with a tapered end for ease of tissue penetration. In some embodiments, the reaction chamber can have any desired configuration accommodated by the needle probe. In some embodiments, the needle probe and the reaction chamber is made of thermally-conducting non-porous material such as silicon, diamond, copper, silicon carbide, graphite, silver, gold, platinum, copper or silicon oxide or alloys or composites thereof, as well as other materials as desired, needed or required. Exemplary material having sufficient heat conductivity for making the reaction chamber or needle probe includes high or relatively high thermal conductivity materials such copper, silver, stainless steel, titanium, aluminum or other materials, alloys, or composite material. Gold, silver, graphene and other materials or alloys can be used as coating material for the reaction chamber or the shaft. The proximal end of the needle probe is coupled to a chemical reactant fluid path or conduit extending from the chemical reactant storage, such as a container.

According to one aspect, the portion of the needle probe including the reaction chamber is configured to contact a target tissue. In some embodiments, the needle and chamber is made of a high thermal conductivity material, where quick temperature regulation is desired. Exemplary material having sufficient heat conductivity for making the reaction chamber includes high or relatively high thermal conductivity materials, such copper, silver, stainless steel, titanium, aluminum or other materials, alloys, or composite material. Gold, silver, graphene and other materials or alloys can be used as coating material for the reaction chamber. In certain other embodiments, the probe and reaction chamber may also be constructed of any material, such as but not limited to radio opaque material, capable of being used for radiography or fluoroscopy purposes. Any desirable combination of toughness, ability to slide or be inserted into tissue, or flexibility and other desirable characteristics for the needle probe is within the spirit of the invention.

The shape and dimension of the reaction chamber according to the present disclosure can vary as desired. In certain embodiments, the reaction chamber can be spherical or cylindrical or square or other desired shape or volume.

The reaction chamber provides heating or cooling effects to a target tissue from the chemical reaction. The proximal end of the needle probe communicates with conduits directly or via extension members. Chemical reactants are provided at various rates, concentrations, temperatures or pressures to the reaction chamber whose shape and/or size affect the level, duration, dimension and shape of the heating or cooling zone of the target tissue.

The size and dimension of the needle shaft according to the present disclosure can vary as desired. In certain embodiments, the length of the needle shaft depending on the location of tissue from the body surface can be in the range of, for example, from 0.1 cm to 200 cm, 0.1 cm to 20 cm or 0.1 cm to 10 cm. For example, a 20 cm long shaft can be fashioned for easily accessible tissue such as the prostate gland and up to 200 cm long shaft can be fashioned for endoscopic procedure to distal lesion of the aerial, or digestive tract. In some embodiments, the diameter of the inner lumen of the needle shaft can be in the range of, for example 100 nanometers to 5 millimeters. According to one aspect, the diameter of the inner lumen can vary along the length of the shaft.

According to one aspect, the needle is compatible with imaging and guiding techniques known to those of skill in the art, which include X-ray, visible light, infrared, ultraviolet, radio frequency, or acoustic waves or ultrasound to image and guide the needle to be placed in the target tissue. During the thermal treatment, thermal sensors, light sensors, and other sensors may be used to monitor and control the treatment. The proximal end of the needle probe according the present disclosure may be equipped with penetration stopping mechanisms to control the depth of penetration of distal end reaction chamber tip. Penetration stopping mechanisms are known to one skilled in the art. For example, the outer surface of the needle shaft may bear markings at known distances. It should be understood that during positioning of the needle probe equipped with the penetration stopping mechanism, the position of the needle probe can be secured as desired during the deployment of an operation.

In some embodiments, the distal end of the needle probe may be steerable by conventional means. Additionally or alternatively, the distal end may be of a flexibility, toughness, and formability differing from that of shaft member. The tip of the distal end can be blunt or tapered or round, but may be of any desirable shape, flexibility and openness like pointed, sharp, cutting, fully open, pre-bent, or bendable, and/or steerable. Such metal as stainless steel, titanium, platinum or metallic alloy, or memory material like nitinol may be used for its structure. In certain embodiments, the proximal end of the needle probe can be connected to extension members comprising separate tubings with each tubing corresponds to an inner lumen and a specific chemical reactant. The tubing, extension member and inner lumen fluidly communicate with opening ports located at the distal end of the needle probe shaft.

In some embodiments, the reaction chamber at the distal end of the needle probe is optionally equipped with a sensor or sensing member (not shown). In other embodiments, the needle probe is equipped with fluid dispensing, aspiration, and/or injection capability through inner lumens and opening ports.

In some embodiments, the needle probe according to the present disclosure as described herein may also include an outer sheath member or guide member coaxially aligned with the needle shaft. Although not limiting the sheath to specific uses, it is contemplated that the outer sheath member may be used for direct injection or circulation of fluids (thermal, chemical, or the like), as an energy delivery probe (thermal energy deposition and/or deprivation, etc.), as a needle or sensor guide, as a sterile cover for a non-sterile probe, to provide for tissue sampling prior or subsequent to a procedure, or combinations thereof. The outer sheath member may also be designed to provide for visualization of relative positioning of each element and for assessing depth immersion into a tissue.

The outer sheath member can be constructed from, or coated with, any material known to one of skill in the art, including thermal conducting materials (such as a metallic material or thermoplastic elastomer), plastics (natural, synthetic), porous or non-porous materials, flexible or rigid materials, or the like as well as a combination of materials arranged in multiple layers. The outer sheath member material can also be manufactured from any materials that are biocompatible and that can be sterilized, therefore being used as a sterile cover for a non-sterile probe. The outer sheath member can be used as a simple guide for the needle probe device and/or having similar capabilities as the needle probe itself. The outer sheath member may also be designed to provide visualization of relative positioning of the outer sheath member, or the needle probe device relative to each other and for assessing depth immersion into a tissue, through the incorporation of any type of material known to one of skill in the art which provides visualization, including laser guidance, optical guidance, ultrasound guidance, radiographic guidance, fluoroscopic guidance, or the like.

The outer sheath member may be manufactured to any thickness necessary and accommodate any size probes, needles, catheters, cannulas, instruments, including energy deposition or deprivation devices such as resistance, microwave, radiofrequency heating, or the like. The outer sheath member can also be designed to contain the same contour or shape of the needle probe tip, whether the tip is pointed, blunt, or any other variations. The outer sheath member can also be designed to accommodate sensors, such as thermal, impedance, pressure, or the like. In one embodiment, the needle probe may be fashioned to contain a tight fit with the coaxial outer sheath.

It should be appreciated that the chemical reactants should be compatible with the other materials that make up the device so they will not react chemically to create undesirable solid, liquid, or gas or cause other deleterious effects. Further, as an example, the chemical reactant is fluid and may be any liquid or gas.

It should be appreciated that the elongated shaft member of the needle probe may be configured in a variety of rigidity and contours without limiting the ability of the probe to thermally modulate the desired target region. The inner lumen may for example but not limited thereto, be a channel, such as a micro- or nano-channel. The length or the diameter of the inner lumen passage may be designated as desired, needed or required. Although, not expressly illustrated, the lumen, passage or channel may have a cylindrical shape or rectangular shape with designated width, W, and area, A, as desired, needed or required. Any of the aforementioned dimensions may increase above or below the micro size magnitude. Additionally, any of the passages may include a variety of shapes and contours as required, needed or desired. They may have a variety of angles or pitches. The passages may be, for example but not limited thereto, a channel such as a nano-channel. Without being bound by any limitations, various embodiments may have lumens (e.g., passages or channels) having the following dimensions: the width, W, may range from about 100 nanometers to 100s of microns; the length, L, may range from about 1 micron to 100 centimeters; the height, H, may range from about 5 microns to 5 millimeters, or the diameter D, may range from about 5 microns to 5 millimeters. It should be appreciated that the dimensions may increase or decrease as desired, needed or required, and these suggested ranges are merely illustrative. For example, the width, W, could range from about 10 nanometers to 10 millimeters. For example, the length, L, may range from 100 nanometers to 1,000 centimeters—or could be greater than 1,000 centimeters. For example, the height, H, may range 100 nanometers to is or 10s of centimeters. Any of these dimensions are applicable to any of the passages indifferent of the structure of the elongated members (shape, angles, contours) that define the passages; as the passages (defined by the elongated members) may be a variety of configurations such as protrusions, walls, panels, pins, posts, or rods, or any combination thereof. The dimensions may vary between respective passages relative to one another. Moreover, the dimensions may vary within a given passage itself. The regions of the passages may vary within the device. Again, these dimensions are merely illustrative and may be increased or decreased as desired or required.

### III. Chemical Reactant Supply Component

The chemical reactant supply component according to the present disclosure provides chemical reactants for the probe. In one embodiments, different chemical reactants are stored in separate containers. In some embodiments, dispensers may be included to provide various concentrations of the chemical reactants as desired. In exemplary embodiments, liquid chemical reactants of pre-determined concentration are stored in the containers. The chemical reactant supply component according to the present disclosure also includes conduits, such as tubes. The tubes operatively connect the chemical reactants to the needle probe. In some embodiments, the conduits are fashioned to connect to the proximal end of the needle probe where the liquid chemical reactants travel via the conduit to the proximal end of the needle probe and pass into one or more inner lumens of the needle probe and reach the interior of the reaction chamber. In other embodiments, the conduits are fashioned to extend via one or more lumens to the distal end of the needle probe where the liquid chemical reactants travel and directly reach the interior of the reaction chamber. According to one aspect, the one or more lumens and conduits each corresponds to inflow of one or more chemical reactants, or to outflow of one or more chemical reactants or reaction products including vapor.

According to one aspect, the chemical reactants comprise chemicals that will produce an exothermal reaction when they meet. An exothermic reaction is a reaction that releases energy into the environment in the form of, e.g., heat or light. More energy is released making chemical bonds than is used breaking them. In an exothermic reaction, the enthalpy change has a negative value: ΔH<0. Examples of chemical exothermic reactions include, e.g., a neutralization reaction, a reaction between water and calcium chloride, a reaction between sodium sulfite and bleach (dilute sodium hypochlorite), a reaction between sodium and chlorine to make sodium chloride (table salt), a reaction between water and any strong acid, a reaction between water and any anhydrous salt, dissolving laundry detergent in water, adding water to anhydrous copper (II) sulfate, formation of ion pair, burning sugar, dehydrating sugar with sulfuric acid, an acid base reaction for example HCl and NaOH at different concentrations. The reaction may be controlled by varying concentration of the reactants and flow rate into the reaction chamber. The reaction chamber may be designed with a static or active mixing element to control mixing and the mixing ratio which in turn controls the rate of heat release into or absorption from the target tissue. The present disclosure contemplates the use of two reactants to create an exothermic reaction. The present disclosure also contemplates the use of three reactants to create an exothermic reaction or two or more or three or more or four or more reactants to create an exothermic endothermic reaction. Accordingly, the present disclosure contemplates using a plurality of reactants to create an exothermic reaction. The present disclosure further contemplates using one or more additional components, such as a catalyst, to regulate the reaction, such as speeding up the reaction or slowing down the reaction. Also, the surface of the chemical reactor can be coated with one or more materials, such as a catalyst, to speed up or reduce the rate of reaction.

According to one aspect, the chemical reactants comprise chemicals that will produce endothermal reaction when they meet. An endothermic reaction is the opposite of an exothermic reaction. Heat is absorbed in an endothermic reaction. In an exothermic reaction, the enthalpy change has a positive value: ΔH>0. Examples of chemical endothermic reactions include, e.g., dissolving a salt, dissolving ammonium chloride in water, mixing water and ammonium nitrate, mixing water with potassium chloride, reacting ethanoic acid with sodium carbonate. The reaction may be controlled by varying concentration of the reactants and flow rate into the reaction chamber. The reaction chamber may be designed with a static or active mixing element to control mixing and the mixing ratio which in turn controls the rate of heat absorption from the target tissue. The present disclosure contemplates the use of two reactants to create an endothermic reaction. The present disclosure also contemplates the use of three reactants to create an endothermic reaction or two or more or three or more or four or more reactants to create an endothermic reaction. Accordingly, the present disclosure contemplates using a plurality of reactants to create an endothermic reaction. The present disclosure further contemplates using one or more additional components, such as a catalyst, to regulate the reaction, such as speeding up the reaction or slowing down the reaction. Also the surface of the chemical reactor can be coated with one or more materials, such as a catalyst, to speed up or reduce the rate of reaction.

The chemical reactant supply component according to the present disclosure also includes a flow control system. In certain embodiments, the flow control system can be connected to a pressurized fluid source, e.g., a syringe or a pump (not represented) well known to one of ordinary skill in the field. The pressurized fluid source provides a means to regulate the rate and pressure of the chemical reaction fluid of interest and also provides a pressurized fluid for removal of reaction products from within the reaction chamber and the needle probe. The chemical reactant supply system may also include a temperature sensing or monitoring system. According to one aspect, one or more temperature sensors may be positioned at the tip of the needle to provide a signal for the control system to control the flow and/or concentration of each reactant into the reaction chamber.

In some embodiments, connectors are deployed at proximal ends of conduits or tubings to allow for disconnection of the needle probe that is readily interchangeable and may be provided as a disposable element according to the present disclosure.

In some embodiments, the proximal end of tubing connections to a vacuum source, e.g., a syringe or a pump (not represented) well known to one of ordinary skill in the field. The vacuum source provides means to aspirate, at a controlled rate and pressure, at least one fluid of interest.

It is contemplated that conduits, tubings and their opening ports can be used interchangeably as injection and/or aspiration members if they are connected to the corresponding vacuum and pressurized sources that can be used sequentially or simultaneously, e.g., through a manifold. For example, a 4-way manifold (not detailed here) could allow for a multiplicity of operations, including but not limited to aspirating fluids and waste, injecting, separately or in conjunction, composition and/or other desirable agents, such as but not limited to chemical reactant liquid, washing solution, or sterilization solution. Moreover, a single tubing can be used for injection and aspiration and washing.

The flow control system according to the present disclosure may include a controller. In certain embodiments, the controller includes modules for converting signals into readable parameters, such as, but not limited to, temperature or resistivity of the target zone and tissue interface, for monitoring and controlling operation of the needle probe. For example, the controller may include indicators for temperature indicator, timer and/or setting for chemical reaction duration, pressure regulator, pressure gauge, power switch. Parameters can be displayed on a controller screen and/or on a separate display screen such as a computer using specific software. Controller may also display reaction chamber temperature, time, initiation or termination of chemical reaction, pressure within reaction chamber and conduit or tubing.

In some embodiments, the wetting and/or non-wetting properties of the materials used in the device ensure proper flow of the liquid chemical reagents and reaction products into and out of the reaction chamber. It should be appreciated that the wetting/non-wetting coatings and/or substrate material of the structure itself may include any portion to be applied on the designated location of the device (needle probe, elongated shaft member, reaction chamber or conduits) as desired, needed or required. It should be appreciated that the wetting and non-wetting properties may be provided by coating materials or by the inherent properties of the substrate materials used to construct the relevant portions of the device. It should also be appreciated that the chemical reactants should be compatible with the needle probe, elongated shaft member, reaction chamber or conduits or any coating materials used so that they will not react chemically to create unwanted waste or cause other deleterious effects.

Examples of materials suitable as wetting coating or wetting substrate include, but are not limited to: hydrophilic materials, particularly when water is used with the chemical reactants; and lyophilic materials, particularly when a fluid other than water is used with the chemical reactants. Examples of materials suitable as non-wetting coating or non-wetting substrate include, but are not limited to: hydrophobic materials, particularly when water is used as working fluid 5; and lyophobic materials, particularly when a fluid other than water is used as working fluid 5. Examples of materials suitable for use as hydrophilic/wetting materials may include, but not limited thereto the following: Metals, glass, ceramic, Silicon, Silicon Carbide, and Diamond, for particular group of chemical reactants. Examples of materials suitable for use as hydrophobic/non-wetting include, but not limited thereto: certain polymers, halogenated hydrocarbons, or chemically altered surfaces of the metals. It should be noted that wetting characteristics are defined for a liquid-solid pair. In an approach, it should be noted that the exact wetting characteristics of a particular embodiment may be determined by the specific interaction between a chosen chemical reactant and chosen wetting coating and/or wetting substrate surface (material) of the needle probe, elongated shaft member, reaction chamber or conduits. Thus, for example, a chemical reactant liquid and wetting coating can be selected jointly according to the exact wetting properties of the liquid-solid pair.

It should be appreciated that the embodiments of the device as disclosed herein may have various other components fabricated using commercially available practices, such as but not limited there to the following: photolithography, micromachining, patterning, etching, ion etching, deep reactive ion etching, plasma etching, laser etching, lithography, and milling. Other available techniques that are included in the context of the various embodiments of the invention include: soldering, brazing, welding, gluing. Moreover, any available coupling, adjoin, and securing techniques and securing structure/systems may be implemented as well within the context of practicing the various embodiments of the invention. Other components or systems may include substrates, chips, sealant, terminals for external connections—as well as others components necessary for the fabrication.

It should be appreciated that the embodiments of the flow control system as disclosed herein may utilize pumps for the fluid (as well as vapor), such as but not limited thereto, the following: electromechanical (e.g., MEMS-based) or electro-osmotic pumps (also referred to as “electric kinetic” or E-K″ pumps).

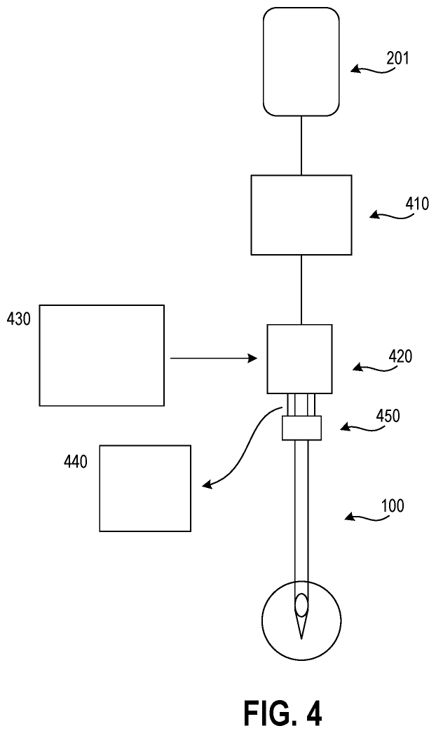

As illustrated in FIG. 4, in certain embodiments, the chemical reactants flow from the container (210) via a conduit that extends from the container to tissue penetrating needle probe (100) where it ultimately reaches the interior of the reaction chamber positioned at the distal end of the needle probe. Values can be included. In one embodiment, a supply valve (410) can be disposed along the conduit to help control any fluid overflow condition, with the supply valve typically comprising a solenoid or other valve controlled by signals from controller circuit. In another embodiment, a controller (430) may also provide control signals to an exhaust valve (440) in response to temperature or fluid pressure signals, typically so as to control a temperature of the reaction chamber at the distal end of the needle probe and/or a pressure of the fluid chemical reactants therein. In some embodiments, a connector (450) is included at the proximal end of the needle probe for directing outflow of waste to the exhaust valve. In alternative embodiments, the controller may also control operation of an applicator (420), such as by varying chemical reactants supplied to the reaction chamber in response to a temperature of a temperature-engaging surface of the reaction chamber or a temperature of the engaged tissue or the like. In one embodiment, the controller may transmit signals for other applicators so as to control a flow of fluid from the applicator, for example, by energizing a pump, actuating a valve, or the like.

To control any overflow of chemical reactant fluid into or through the needle probe, the supply valve along the conduits may be pulsed so as to allow sufficient flow during different portions of the treatment.

### IV. Tissue Substrates

Device and method according to the present disclosure rely on placement and use of one or more needle probes positioned at or within a treatment region of a patient. The treatment region may be located anywhere in the body where needle probe treatment may be beneficial. Tissue to be treated may be cancerous or noncancerous. Most commonly, but certainly not limiting, the treatment region will comprise a solid tumor within an organ of the body, such as the liver, kidney, lung, bowel, stomach, pancreas, breast, prostate, uterus, muscle, brain. The volume of fluid-based chemical reactants to be injected depends on the size of the tumor or other lesion, typically having a total volume from 1 mm³ to 150 cm³, 5 mm³ to 100 cm³, 5 mm³ to 2 cm³, usually from 1 cm³ to 50 cm³, and often from 2 cm³ to 35 cm³. The peripheral dimensions of the treatment region may be regular, e.g. spherical or ellipsoidal. However, typical tumor shape is irregular. The treatment region may be identified using conventional imaging techniques capable of elucidating a target tissue, e.g. tumor tissue, such as ultrasonic scanning, magnetic resonance imaging (NMI), computer-assisted tomography (CAT), fluoroscopy, nuclear scanning (using radio labeled tumor-specific probes), and the like. High-resolution ultrasound or CT can be employed to monitor the size and location of the tumor or other lesion being treated, intraoperatively or externally.

The targeted site can include tissues of a sinus, a nasal passageway, an oral cavity, a blood vessel, an arteriovascular malformation, a heart, an airway, a lung, a bronchus, a bronchiole, a lung collateral ventilation pathway, a larynx, a trachea, a Eustachian tube, a uterus, a vaginal canal cervical tissue, a fallopian tube, a liver, an esophagus, a tongue, a stomach, a duodenum, an ileum, a colon, a rectum, a bladder, a prostate, a urethra, a ureter, a vas deferens, a kidney, a gall bladder, a pancreas, a bone, an interior of a bone, a joint capsule, a tumor, a plexus of dilated veins, a fibroid, a neoplastic mass, brain tissue, skin, lymph nodes, sweat glands, adipose tissue, a keloid, scar tissue, muscle tissue, epidermal tissue, connective tissue, hyperplastic tissue, hypertrophic tissue, an ovary, a wart, a cyst, a cornea and a retina.

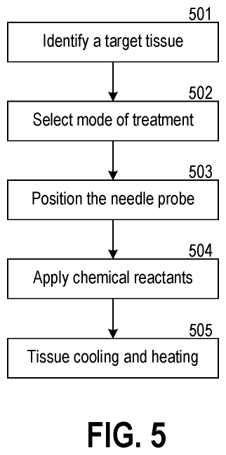

As illustrated in FIG. 5, an appropriate target tissue is identified (501), such as a tumor, nerve, muscle, neuromuscular junction, connective tissue, adipose tissue layer, or the like ct. A treatment mode (502) such as heating or cooling may be selected, and the needle probe is positioned (503). Positioning of the needle probe may, for example, comprise inserting one or more tissue-penetrating needle probes into the target tissue, engaging the skin surface with a skin-engaging surface of a handpiece securing the needle probe or probes, and/or the like. In alternative embodiments, injury to the skin may be inhibited, such as by warming the skin surface, infusing a warmed biocompatible fluid such as saline, applying a cryoprotectant such as DMSO, or the like.

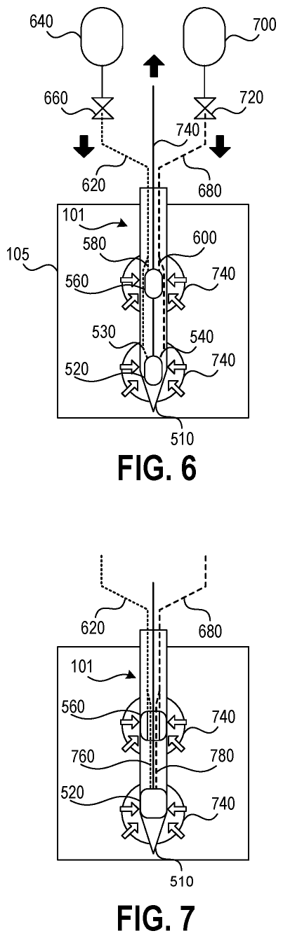

FIG. 6 depicts a schematic illustration of a probe having two reaction chambers. FIG. 6 shares common elements with the embodiment of FIG. 1. In FIG. 6, a probe or needle 101 is depicted within a given volume of target tissue 105. The probe/needle 101 includes a sharpened tip 510 which is also shown in FIG. 1. A first reaction chamber 520 is positioned at or above the sharpened tip 510. A first conduit 530 and second conduit 540 is connected to the first reaction chamber 520. A second reaction chamber 560 is positioned approximately midway of the probe/needle 101. A first conduit 580 and second conduit 600 is connected to the second reaction chamber 560. As depicted, a first reagent conduit 620 is connected to a first reagent container 640 via a first flow control system 660. A first reagent is provided from the first reagent container 640 via the first flow control system 660 to the second reaction chamber 560 and/or the first reaction chamber 520. As depicted, a second reagent conduit 680 is connected to a second reagent container 700 via a second flow control system 720. A second reagent is provided from the second reagent container 700 via the second flow control system 720 to the second reaction chamber 560 and/or the first reaction chamber 520. As shown in FIG. 6, an outflow conduit 740 connects both the first reaction chamber 520 and the second reaction chamber 560 for outflow of contents of the first reaction chamber 520 and the second reaction chamber 560. It is to be understood that the first reaction chamber 520 and the second reaction chamber 560 may have their own separate outflow conduit. As shown in FIG. 6, a shaded area 740 surrounding each reaction chamber indicates the volume of tissue subject to a temperature change from the reaction chamber. As illustrated in FIG. 6, an endothermic reaction as indicated by the arrows occurs in each reaction chamber thereby drawing in energy or heat from the surrounding tissue leading to cooling of the shaded volume of tissue. It is to be understood that an exothermic reaction can be used to heat the shaded volume of tissue.

FIG. 7 depicts an alternate embodiment of FIG. 6 where each reaction chamber 520 and 560 has a common first conduit 760 and second conduit 780, with FIG. 7 illustrating common structure with FIG. 6.

Chemical reactants may be disposed into the reaction chamber whereupon an exothermic or endothermic chemical reaction takes place to thereby heat or cool the target tissue (504) so as to effect the desired treatment of that tissue. For example, the target tissue may be heated to a temperature where the target tissue is destroyed by killing cells within the target tissue. Such a temperature may be at least 40° C. to 80° C. or higher. For example, the target tissue may be cooled to a temperature where the target tissue is destroyed by killing cells within the target tissue. Such a temperature may be at least 30° C. to −200° C. or lower, such as 0° C. to −50° C., or −4° C. to −20° C. The tissue response and healing (505) may follow immediately after cooling or heating is applied, or may take place over a considerable time (such as when efficacy is achieved through apoptosis or the like). In certain embodiments, if a short duration or trial treatment was performed to verify the target tissue and treatment effect, retreatment may be performed.

### V. Embodiments

Embodiments of the present disclosure include a device for treating target tissue by heating or cooling including a probe component, wherein the probe component comprises an elongated shaft member having one or more inner lumens for passage of one or more chemical reactants operatively coupled to a reaction chamber positioned in the interior of the probe; one or more inner lumens for removal of liquid or vapor from the reaction chamber, and a chemical reactant supply component comprising one or more containers and a flow control system, wherein the probe component is operatively connected to the chemical reactant supply component, wherein the flow control system comprises one or more fluid control regulators to dispense fluid from the one or more containers into the probe component and into the reaction chamber. According to one aspect, the reaction chamber is positioned at the distal end of the probe component. According to one aspect, the device includes an inflow conduit and an outflow conduit operatively connected to the reaction chamber and a pump to deliver wash fluid into the inflow conduit, the reaction chamber and the outflow conduit. According to one aspect, the flow control system is configured to control the rate and direction of the flow of chemical reactants from the chemical reactant supply component to the reaction chamber. According to one aspect, the probe component has a pointed closed tip. According to one aspect, the probe component has a blunt closed tip.

Embodiments of the present disclosure include a method for thermal modulation of a target tissue including providing a probe device having a reaction chamber therein in contact with the target tissue, and applying one or more chemical reactants to the reaction chamber, wherein the chemical reactants react in the reaction chamber resulting in heating or cooling of the target tissue. According to one aspect, the one or more chemical reactants can be delivered simultaneously or sequentially. According to one aspect, the one or more chemical reactants and reaction products are removed from the reaction chamber after a predetermined time. According to one aspect, a wash step is included prior to, during or after the application of one or more chemical reactants. According to one aspect, one or more conduits are used to deliver one or more chemical reactants or reaction products. According to one aspect, heat is released via exothermal reaction in the reaction chamber to heat the target tissue. According to one aspect, heat is absorbed via endothermal reaction in the reaction chamber to cool the target tissue. According to one aspect, the device includes a flow control system to control the rate and direction of the flow of the chemical reactants. According to one aspect, heating and cooling profiles are controlled via regulating the kind, amount and mixing ratio of the chemical reactants and flow rate and direction of the chemical reactants. According to one aspect, the tissue can be heated or cooled depending on the chemical reactants. According to one aspect, the tissue can be heated or cooled repeatedly.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

Those having skill in the art, with the knowledge gained from the present disclosure, will recognize that various changes can be made to the disclosed devices and methods in attaining these and other advantages, without departing from the scope of the present invention. Accordingly, it should be understood that the features described herein are susceptible to changes or substitutions. The specific embodiments illustrated and described herein are for illustrative purposes only, and not limiting of the invention as set forth in the appended claims.

Aspects of the present technology are described below, and various examples of the present technology are described as numbered clauses (1, 2, 3, etc.) for convenience. These clauses are provided as examples and do not limit the present technology. It is noted that any of the dependent clauses may be combined in any combination, and placed into a respective independent clause. The other clauses can be presented in a similar manner.

1. A device configured to thermally modulate a target region of a patient, comprising
a probe including—
   an elongated shaft member having a proximal end portion and a distal end portion,
   a reaction chamber,
   a plurality of lumens each extending from the proximal end of the elongated shaft to the reaction chamber, the plurality of lumens including a first lumen and a second lumen, and
   a tip at a distal terminus of the probe; and
a chemical reactant supply fluidically coupled to the proximal end portion of the probe and including a first container and a second container, wherein the first container is fluidically coupled to the first lumen of the probe and configured to contain a first chemical reactant, and wherein the second container is fluidically coupled to the second lumen of the probe and configured to contain a second chemical reactant,
wherein the reaction chamber is configured to receive and enable mixing of the first chemical reactant and the second chemical reactant to cause an exothermic or endothermic reaction, and
wherein the tip is configured to penetrate tissue of a patient such that the reaction chamber is positioned adjacent a target region beneath a skin surface of the patient.

2. The device of any one of the clauses herein, wherein the plurality of lumens includes a third lumen extending from and fluidically coupled to the reaction chamber, wherein the third lumen is configured to relieve pressure from the reaction chamber.

3. The device of any one of the clauses herein, further comprising a flow control system comprising one or more regulators configured to regulate flow of the first chemical reactant from the first container.

4. The device of any one of the clauses herein, further comprising a flow control system including one or more regulators configured to regulate flow of the first chemical reactant from the first container independent of flow of the second chemical reactant from the second container.

5. The device of any one of the clauses herein, further comprising a flow control system including one or more valves configured to regulate flow of the first chemical reactant from the first container based on an expected rate of temperature change associated with mixing the first chemical reactant and the second chemical reactant within the reaction chamber.

6. The device of any one of the clauses herein, wherein the tip is configured to penetrate tissue such that the reaction chamber and a portion of the elongated shaft is positioned beneath the skin surface.

7. The device of any one of the clauses herein, wherein the tip is a closed tip.

8. The device of clause 7, wherein the tip is tapered such that a distal terminus of the tip is pointed.

9. The device of any one of the clauses herein, wherein the reaction chamber is one of a plurality of reaction chambers of the probe.

10. The device of any one of the clauses herein, wherein the reaction chamber is positioned distal to the distal end portion of the elongated shaft member.

11. The device of any one of the clauses herein, wherein the first lumen and the second lumen are parallel to one another and extend along a longitudinal axis of the probe.

12. The device of any one of the clauses herein, further comprising an electrical impedance sensor at or distal to the distal end portion of the probe.

13. The device of any one of the clauses herein, further comprising one or more sensors including at least one of an electrical impedance sensor, a temperature sensor, or a pressure sensor, wherein at least one of the one or more sensors is positioned proximal to the reaction chamber.

14. The device of any one of the clauses herein, wherein a portion of the elongated shaft includes an outer surface coated with or comprising a material configured to inhibit dispersion of heat therefrom.

15. The device of any one of the clauses herein, wherein the reaction chamber comprises copper, silver, stainless steel, titanium, or aluminum.

16. The device of any one of the clauses herein, wherein mixing the first chemical reactant and the second chemical reactant causes an exothermic reaction, and wherein, in operation, the exothermic reaction cools the target region.

17. A method for thermally modulating a target region of a patient, comprising:
providing a device including a probe and a chemical reactant supply fluidically coupled to the probe, wherein the probe includes an elongated shaft member having a proximal end portion and a distal end portion, a reaction chamber, a lumen extending from the proximal end to the reaction chamber, and a closed tip at a distal terminus of the probe, and wherein the chemical reactant supply contains a first chemical reactant and a second chemical reactant fluidically separate from the first chemical reactant;
positioning the probe such that the reaction chamber is adjacent a target region beneath a skin surface of a patient; and
after positioning the probe, causing the first chemical reactant and the second chemical reactant to flow through the lumen and be mixed in the reaction chamber to cause an exothermic or endothermic reaction, thereby thermally modulating the target region.

18. The method of any one of the clauses herein, wherein the chemical reactant supply includes a first container containing the first chemical reactant and a second container containing the second chemical reactant, wherein causing the first chemical reactant and the second chemical reactant to be mixed in the reaction chamber comprises:
causing the first chemical reactant to flow from the first container to the reaction chamber via the lumen; and
causing the second chemical reactant to flow from the second container to the reaction chamber via the lumen.

19. The method of any one of the clauses herein, further comprising regulating the flow of the first chemical reactant based on a temperature signal received from a sensor of the device.

20. The method of any one of the clauses herein, further comprising receiving an electrical impedance signal from a sensor of the device, wherein positioning the probe is based on the electrical impedance signal received from the sensor.

21. A device configured to thermally modulate a target region of a patient, comprising
a probe including—
   an elongated shaft member having a proximal end portion and a distal end portion,
   a reaction chamber,
   a lumen extending from the proximal end to the reaction chamber, and
   a tip at a distal terminus of the probe; and
a chemical reactant supply fluidically coupled to the probe at the proximal end portion and including a first container and a second container, wherein the first container is configured to contain a first chemical reactant and the second container is configured to contain a second chemical reactant different than the first chemical reactant, the first container and the second container being fluidically coupled to the lumen;
wherein the reaction chamber is configured to receive via the lumen the first chemical reactant and the second chemical reactant to cause an exothermic or endothermic reaction, and
wherein the tip is configured to penetrate tissue such that the reaction chamber is positioned adjacent a target region beneath a skin surface.

22. A device for treating target tissue by heating or cooling, comprising
a probe component, wherein the probe component comprises an elongated shaft member having one or more inner lumens for passage of one or more chemical reactants operatively coupled to a reaction chamber positioned in the interior of the probe;
one or more inner lumens for removal of liquid or vapor from the reaction chamber, and
a chemical reactant supply component comprising one or more containers and a flow control system,
wherein the probe component is operatively connected to the chemical reactant supply component, wherein the flow control system comprises one or more fluid control regulators to dispense fluid from the one or more containers into the probe component and into the reaction chamber.

23. The device of clause 22 wherein the reaction chamber is positioned at the distal end of the probe component.

24. The device of clause 22 wherein the device includes an inflow conduit and an outflow conduit operatively connected to the reaction chamber and a pump to deliver wash fluid into the inflow conduit, the reaction chamber and the outflow conduit.

25. The device of clause 22 wherein the flow control system is configured to control the rate and direction of the flow of chemical reactants from the chemical reactant supply component to the reaction chamber.

26. The device of clause 22 where the probe component has a pointed closed tip.

27. The device of clause 22 wherein the probe component has a blunt closed tip.

28. A method for thermal modulation of a target tissue comprising providing a probe device having a reaction chamber therein in contact with the target tissue, and applying one or more chemical reactants to the reaction chamber, wherein the chemical reactants react in the reaction chamber resulting in heating or cooling of the target tissue.

29. The method of clause 28 wherein the one or more chemical reactants can be delivered simultaneously or sequentially.

30. The method of clause 28 wherein the one or more chemical reactants and reaction products are removed from the reaction chamber after a predetermined time.

31. The method of clause 28 wherein a wash step is included prior to, during or after the application of one or more chemical reactants.

32. The method of clause 28 wherein one or more conduits are used to deliver one or more chemical reactants or reaction products.

33. The method of clause 28 wherein heat is released via exothermal reaction in the reaction chamber to heat the target tissue.

34. The method of clause 28 wherein heat is absorbed via endothermal reaction in the reaction chamber to cool the target tissue.

35. The method of clause 28 wherein the device includes a flow control system to control the rate and direction of the flow of the chemical reactants.

36. The method of clause 28 wherein heating and cooling profiles are controlled via regulating the kind, amount and mixing ratio of the chemical reactants and flow rate and direction of the chemical reactants.

37. The method of clause 28 wherein the tissue can be heated or cooled depending on the chemical reactants.

38. The method of clause 28 wherein the tissue can be heated or cooled repeatedly.

What is claimed is:

1. A device configured to thermally modulate a target region of a patient, comprising
a probe including—
an elongated shaft member having a proximal end portion and a distal end portion,
a reaction chamber,
a plurality of lumens each extending from the proximal end of the elongated shaft to the reaction chamber, the plurality of lumens including a first lumen and a second lumen, and
a tip at a distal terminus of the probe; and
a chemical reactant supply fluidically coupled to the proximal end portion of the probe and including a first container and a second container, wherein the first container is fluidically coupled to the first lumen of the probe and configured to contain a first chemical reactant, and wherein the second container is fluidically coupled to the second lumen of the probe and configured to contain a second chemical reactant,
wherein the reaction chamber is configured to receive and enable mixing of the first chemical reactant and the second chemical reactant to cause an exothermic or endothermic reaction, and
wherein the tip is configured to penetrate tissue of a patient such that the reaction chamber is positioned adjacent a target region beneath a skin surface of the patient.

2. The device of claim 1, wherein the plurality of lumens includes a third lumen extending from and fluidically coupled to the reaction chamber, wherein the third lumen is configured to relieve pressure from the reaction chamber.

3. The device of claim 1, further comprising a flow control system comprising one or more regulators configured to regulate flow of the first chemical reactant from the first container.

4. The device of claim 1, further comprising a flow control system including one or more regulators configured to regulate flow of the first chemical reactant from the first container independent of flow of the second chemical reactant from the second container.

5. The device of claim 1, further comprising a flow control system including one or more valves configured to regulate flow of the first chemical reactant from the first container based on an expected rate of temperature change associated with mixing the first chemical reactant and the second chemical reactant within the reaction chamber.

6. The device of claim 1, wherein the tip is configured to penetrate tissue such that the reaction chamber and a portion of the elongated shaft is positioned beneath the skin surface.

7. The device of claim 1, wherein the tip is a closed tip.

8. The device of claim 7, wherein the tip is tapered such that a distal terminus of the tip is pointed.

9. The device of claim 1, wherein the reaction chamber is one of a plurality of reaction chambers of the probe.

10. The device of claim 1, wherein the reaction chamber is positioned distal to the distal end portion of the elongated shaft member.

11. The device of claim 1, wherein the first lumen and the second lumen are parallel to one another and extend along a longitudinal axis of the probe.

12. The device of claim 1, further comprising an electrical impedance sensor at or distal to the distal end portion of the probe.

13. The device of claim 1, further comprising one or more sensors including at least one of an electrical impedance sensor, a temperature sensor, or a pressure sensor, wherein at least one of the one or more sensors is positioned proximal to the reaction chamber.

14. The device of claim 1, wherein a portion of the elongated shaft includes an outer surface coated with or comprising a material configured to inhibit dispersion of heat therefrom.

15. The device of claim 1, wherein the reaction chamber comprises copper, silver, stainless steel, titanium, or aluminum.

16. The device of claim 1, wherein mixing the first chemical reactant and the second chemical reactant causes an exothermic reaction, and wherein, in operation, the exothermic reaction cools the target region.

17. A method for thermally modulating a target region of a patient, comprising:
providing a device including a probe and a chemical reactant supply fluidically coupled to the probe, wherein the probe includes an elongated shaft member having a proximal end portion and a distal end portion, a reaction chamber, a lumen extending from the proximal end to the reaction chamber, and a closed tip at a distal terminus of the probe, and wherein the chemical reactant supply contains a first chemical reactant and a second chemical reactant fluidically separate from the first chemical reactant;
positioning the probe such that the reaction chamber is adjacent a target region beneath a skin surface of a patient; and
after positioning the probe, causing the first chemical reactant and the second chemical reactant to flow through the lumen and be mixed in the reaction chamber to cause an exothermic or endothermic reaction, thereby thermally modulating the target region.

18. The method of claim 17, wherein the chemical reactant supply includes a first container containing the first chemical reactant and a second container containing the second chemical reactant, wherein causing the first chemical reactant and the second chemical reactant to be mixed in the reaction chamber comprises:
causing the first chemical reactant to flow from the first container to the reaction chamber via the lumen; and
causing the second chemical reactant to flow from the second container to the reaction chamber via the lumen.

19. The method of claim 17, further comprising regulating the flow of the first chemical reactant based on a temperature signal received from a sensor of the device.

20. The method of claim 17, further comprising receiving an electrical impedance signal from a sensor of the device, wherein positioning the probe is based on the electrical impedance signal received from the sensor.

21. A device configured to thermally modulate a target region of a patient, comprising
a probe including—
an elongated shaft member having a proximal end portion and a distal end portion,
a reaction chamber,
a lumen extending from the proximal end to the reaction chamber, and
a tip at a distal terminus of the probe; and
a chemical reactant supply fluidically coupled to the probe at the proximal end portion and including a first container and a second container, wherein the first container is configured to contain a first chemical reactant and the second container is configured to contain a second chemical reactant different than the first chemical reactant, the first container and the second container being fluidically coupled to the lumen;
wherein the reaction chamber is configured to receive via the lumen the first chemical reactant and the second chemical reactant to cause an exothermic or endothermic reaction, and
wherein the tip is configured to penetrate tissue such that the reaction chamber is positioned adjacent a target region beneath a skin surface.
